(19) United States
(12) Patent Application Publication
(10) Pub. No.: US 2019/0021778 A1
(43) Pub. Date: Jan. 24, 2019

(54) IMAGE-GUIDED REMOVAL AND THERMAL THERAPY OF BREAST CANCER

(71) Applicant: Kambiz Dowlatshahi, Chicago, IL (US)

(72) Inventor: Kambiz Dowlatshahi, Chicago, IL (US)

(21) Appl. No.: 15/894,486

(22) Filed: Feb. 12, 2018

Related U.S. Application Data
(63) Continuation of application No. 14/821,984, filed on Aug. 10, 2015, now abandoned.
Application No. 14/821,984 is a continuation of application No. 13/942,261, filed on Jul. 15, 2013, now abandoned.
Application No. 13/942,261 is a continuation-in-part of application No. 11/753,510, filed on May 24, 2007, now Pat. No. 8,486,127.
(60) Provisional application No. 61/792,127, filed on Mar. 15, 2013.
(60) Provisional application No. 60/915,852, filed on May 3, 2007.
(60) Provisional application No. 60/808,064, filed on May 24, 2006.

(51) Int. Cl. A61B 18/04; A61B 34/20; A61B 18/00; A61B 17/22

(52) U.S. Cl. CPC A61B 18/04; A61B 34/20; A61B 2018/00791; A61B 2018/00333; A61B 2018/0022; A61B 2017/22051; A61B 2018/046; A61B 2018/00577

(57) ABSTRACT

A method of removing undesirable cells and killing cells surrounding a cavity caused by the removal of the undesirable cells includes the step of inserting a probe through an incision made in the skin of a patient. The method further includes the steps of removing undesirable cells using the probe and using image-guided technology and inserting a balloon into the cavity formed by the removal of the undesirable cells. Still further, the method includes the steps of conforming the balloon to a shape of the cavity and providing sufficient fluid to fill the cavity based on the volume and reaching a temperature in the fluid to achieve a temperature in the tissue surrounding the cavity that kills cells surrounding the cavity.

This patent application is a continuation of U.S. patent application Ser. No. 14/821,984 filed on Aug. 10, 2015, which is a continuation of U.S. patent application Ser. No. 13/942,261 filed on Jul. 15, 2013, which claims the benefit of U.S. Provisional Patent Application No. 61/792,127, filed Mar. 15, 2013, and further which is a continuation-in-part of U.S. Ser. No. 11/753,510 filed on May 24, 2007 (now U.S. Pat. No. 8,486,127), which is a non-provisional claiming priority under 35 U.S.C. § 119(e) to U.S. Provisional Patent Application No. 60/808,064, filed May 24, 2006, and U.S. Provisional Patent Application No. 60/915,852, filed May 3, 2007, the entirety of each of which are incorporated herein by reference in their entirety.

### BACKGROUND OF THE INVENTION

Methods, apparatus and compositions are described for treating post-surgical breast cancer by applying heat to tissue surrounding a cavity remaining after surgical removal of cancerous tissue, thereby ablating remaining transformed cells and preventing metastasis of the transformed cells. Applications include use after lumpectomy and other forms of excision of small tumors detected by mammography.

Widespread annual screening mammography has resulted in detection of greater percentages of breast cancers measuring 1 cm in diameter. The diagnosis of malignancy and determination of the prognostic factors are typically made by image guided needle biopsy. The treatment of breast cancers involves a number of possible alternatives, including surgical removal, radiation therapy, chemotherapy, thermotherapy and combinations thereof. Breast tumors are surgically removed generally together with sentinel/regional lymph nodes.

Conventional techniques of post-operative treatment of residual tumors following gross removal of tumors include sequential or simultaneous administration of radiation and chemotherapy. Originally, radiation therapy involved whole breast irradiation. More recently, partial irradiation of the lumpectomy cavity places an inflatable balloon in the space where the cancer was surgically removed to irradiate the surrounding tissue using an Iridium seed at the center of the balloon. This procedure typically requires multiple rounds of irradiation such as 15 minutes exposure, twice a day over a five day period. This procedure is also known as brachytherapy and has become the preferred alternative to whole breast irradiation. The rationale for this treatment is based upon the observation that over 80% of breast cancer recurrences appear within a radius of one centimeter from the initial tumor border. Success rates (reduced incidence of recurrence) using brachytherapy in breast cancer are considered to be similar to those of whole breast irradiation.

Breast cancer is a common malignancy in the United States and elsewhere in the world. Widespread screening mammography has resulted in detection of smaller tumors, in turn leading to breast saving operation i.e. lumpectomy, and irradiation. Partial irradiation of the affected site delivered through a radium source placed at the center of a balloon in one week is replacing the whole breast external beam irradiation given in six weeks. Although this approach shortens the treatment time and encourages more women to seek a breast saving operation, it is still burdensome for the patient to carry a balloon in the breast for a week receiving treatment twice a day. Furthermore, the cost of the breast irradiation therapy remains high; an item which is part of the burgeoning healthcare budget.

Since the initial successes of brachytherapy, various intraoperative therapeutic procedures now utilize the cavity formerly occupied by the bulk of the tumor for placement of an inflatable device for subsequent tumor therapy, often in a combined modality (radiation and/or chemotherapy and/or hyperthermia together).

Radiation sources, e.g. a radium seed may be placed inside a balloon which is implanted into a breast of a patient. The balloon stays in place during the treatment period, e.g. for 10-15 days. When a patient returns for each treatment session (generally daily), the radiation source is inserted into the balloon for a period of time, perhaps twice a day, until the treatment period ends. This is inconvenient for the patient, and has the attendant risks and costs of radiation. Expenses include special facilities, radiation source and technical support.

Heat in balloons has been used to control uterine bleeding but not to destroy transformed cells. The uterus muscular tissue is very different type of tissue than breast and has a different construction than the breast, which has much fatty tissue. Pressures of around 150 mm Hg are employed before the balloon device is activated, although pressure is generally not reported nor are descriptions of pathological effects on tissue of heat delivered in this manner.

### SUMMARY

Methods, apparatus and compositions are disclosed to remove cancerous tissue and ablate at least a 1 cm wide zone of tissue surrounding the site of removal of cancerous tissue, such as a breast tumor, by heating for a short time using a “hot balloon.”

While the methods and compositions disclosed herein may be used for first occurrence of cancer, the methods herein are especially beneficial for recurrence of cancer. In particular, a patient with recurrent breast cancer who has already undergone radiation therapy, is unable to undergo further radiation therapy. For such patients, the only current option is to undergo a mastectomy. The methods and compositions disclosed herein provide therapy for the removal of cancerous tissue, as opposed to a mastectomy.

In contrast to post-surgical radiation therapy of breasts using balloon implants, methods and compositions are disclosed that use a balloon catheter with a balloon configured to conform to surgical cavities in the breast, balloons constructed to fill the cavity in the fatty breast tissue, using materials with sufficient rigidity and pressure to maintain shape of the balloon and to provide sufficient heat to destroy or inactivate cells in a specified thickness of tissue lining and surrounding the cavity.

Thermal therapy applied to breast cavities resulting from breast cancer surgery destroys transformed and potentially transformed tissue after a cancerous breast mass has been surgically removed.

Only one treatment is generally required, no special facilities are needed, and an MRI can determine if cells in a specific area were destroyed or inactivated. Single treatment times of about 15 minutes are contemplated as adequate to remove, kill or inactivate transformed tissue in the vicinity of the post-surgical cavity.

A method of removing undesirable cells and killing cells surrounding a cavity caused by the removal of the undesirable cells, includes the step of inserting a probe through an incision made in the skin of a patient. The method further includes the steps of removing undesirable cells using the probe and using image-guided technology and inserting a balloon into the cavity formed by the removal of the undesirable cells. Still further, the method includes the steps of conforming the balloon to a shape of the cavity and providing sufficient fluid to fill the cavity based on the volume and reaching a temperature in the fluid resulting in a temperature in the tissue surrounding the cavity sufficient to kill cells surrounding the cavity.

A method of removing undesirable cells and killing cells surrounding a cavity caused by the removal of the undesirable cells includes the step of inserting a probe through a small incision made in the skin of a patient. The method further includes the steps of removing undesirable cells using the probe and using image-guided technology and inserting a balloon into the cavity formed by the removal of the undesirable cells. Still further, the method includes the steps of conforming the balloon to a shape of the cavity and providing sufficient fluid to fill the cavity based on the volume and reaching a temperature in the fluid to achieve a temperature in the tissue surrounding the cavity that kills cells surrounding the cavity. The method also includes the step of placing at least one temperature sensor under the skin of the patient and spaced from the cavity.

### BRIEF DESCRIPTION OF THE DRAWINGS

FIG. 1 shows a flow chart of the steps used in the high temperature thermotherapy treatment of breast tissue.

FIG. 2 shows a cross section cartoon of the catheter, balloon and sensors placed in and around a post-surgical breast cavity.

FIG. 3 is a diagrammatic section of a goat mammary gland with a balloon for thermal therapy and a zone for necrosis. Roman numerals indicate areas to be examined.

FIG. 4A shows a picture of a goat mammary gland 5 days post-therapy.

FIG. 4B shows a picture of a goat mammary gland 20 days post-therapy.

FIG. 5 shows a microscopic view of a goat mammary gland.

FIG. 6 shows the depth of necrosis of goat mammary gland upon thermal therapy.

FIG. 7 is a diagram of a hot balloon and treated segments.

FIG. 8 is a flow chart of a controller for operation of a balloon catheter.

FIG. 9 is a set of stereo-tactic images taken of a sheep's udder after depositing powdered eggshell therein.

FIG. 10 is a set of stereo-tactic images taken after a probe for removal of the powdered eggshell simulating cancerous tissue has been inserted into the sheep's udder.

FIG. 11 is a first sample of tissue removed from the sheep's udder using the probe of FIG. 10.

FIG. 12 is a second sample of tissue removed from the sheep's udder using the probe of FIG. 10.

FIG. 13 is a third sample of tissue removed from the sheep's udder using the probe of FIG. 10.

FIG. 14 is a set of stereo-tactic images taken after the probe has removed the powdered eggshell simulating cancerous tissue from the sheep's udder and prior to removal of the probe from the sheep's udder.

FIG. 15 is a set of stereo-tactic images taken after the probe has been removed from the sheep's udder and depicting a cavity left by the removal of the powdered eggshell and the cancerous tissue.

FIG. 16 is a set of stereo-tactic images taken after a catheter has been inserted into the sheep's udder and a balloon associated with the catheter has been inflated to fill the cavity left by removal of the powdered eggshell simulating the cancerous tissue.

FIG. 17 is a set of stereo-tactic images taken after a thermal sensor needle has been placed 1 centimeter from the balloon.

FIG. 18 is a side elevational view of a breast of a patient depicting breast cancer therein.

FIG. 19 is a side elevational view of the breast of FIG. 18 as the breast cancer is removed.

FIG. 20 is a side elevational view of the breast of FIG. 18 after the breast cancer has been removed, leaving a cavity.

FIG. 21 is a side elevational view of the breast of FIG. 18 after a balloon has been inserted into a cavity formed after removal of the breast cancer and depicting a thermal sensor spaced 1 centimeter from the cavity.

FIG. 22 is a side elevational view of the breast of FIG. 18 after the balloon and thermal sensor have been removed, wherein a 1 centimeter zone of ablation is depicted.

FIG. 23 is an MRI image of thermally treated breast cancer illustrating a zone of necrosis around a lumpectomy site.

### DETAILED DESCRIPTION

A method for treating post-surgical breast cavities to ablate, kill, destroy, and/or sterilize any cancerous tissue that remains within a one centimeter radius of the cavity uses a balloon catheter commonly used in various surgical interventions and described for uterine applications in U.S. Pat. Nos. 4,949,718 and 6,139,570.

Partial breast irradiation (PBI) surrounding the lumpectomy cavity, utilizing a balloon with a radiation seed in its center (brachytherapy), has become an alternative to whole breast irradiation. The rationale for this new treatment is based upon the observation that over 80% of the breast cancer recurrences appear within one cm radius of the initial tumor border. Indeed the short term report of patients treated with brachytherapy using a Mammosite balloon indicates similar success rate as the whole breast irradiation. The depth of the targeted breast tissue destroyed by irradiation is one centimeter. A multi-center prospective clinical trial by NSABP is underway to test this concept.

Boiling water scalds the skin, burns the tongue and destroys bacteria. The mechanism of destruction is due to cell membrane disruption, disintegration of the intracellular organelles and the cell nucleus. The essence of the thermal therapy for post-lumpectomy cavity is that high heat (sub-boiling temperatures) penetrates the surrounding breast tissue to a depth comparable to radiation. The devitalized tissue is partially liquefied, which can be percutaneously aspirated, and partially removed by macrophages with the scar development comparable to any healing process.

A method for treating breast cancer reduces transformed tissue remaining in the breast after surgery. A post-surgical method directed to a cavity in a breast causes necrosis of the tissue surrounding the cavity. A method for high temperature thermotherapy of the tissue surrounding a removed breast cancer mass causes necrosis of remaining transformed tissue. Since no reliable in vivo method has been developed to determine if cells surrounding the cavity are transformed, the method targets the entire surface of the cavity, destroying cells uniformly within a targeted radius. This will eliminate any transformed cells or tissue and prevent the recurrence of breast cancer and/or metastases to other tissues.

The method of post-surgical transformed breast tissue requires a single application to ablate the transformed tissue and prevents recurrence of breast cancer or metastases to other tissues.

Thermotherapy is determined to be sufficient by itself to eliminate remaining cancerous cells in breast tissue after surgical removal. High temperature breast cancer removal using laser treatment has demonstrated that a temperature of 60° C. (140° F.) is sufficient to destroy breast tumors. Experience with such treatments has led to methods described herein whereby thermotherapy using hot fluid is sufficient to destroy post-surgical cavity tissue. The need for radioactive compounds, toxic chemicals, lasers, special fluids or gases is eliminated, because heated 5% Dextrose saline provides sufficient thermal transport and heat diffusion to the tissue surrounding the cavity and is capable of destroying tissue within a two centimeter radius of the balloon diameter. The need for multiple treatments using radiation therapy alone is eliminated, a single treatment to ablate tissue surrounding the resection cavity. The liquid can be maintained at the correct temperature and maintained at that temperature with a heating coil used in the balloon for the requisite time. Surface tissue damage is easily avoided by using a cold pack on the skin above the treatment area. Using the proper balloon or bladder will further allow the fluid to conform to non-uniform resection cavity and provide even heating in a uniform radius no matter the cavity shape.

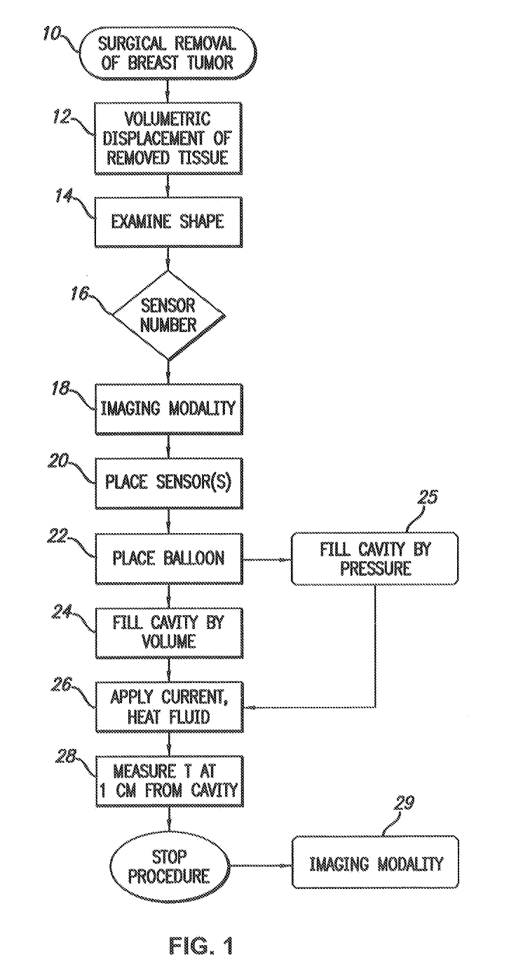

Referring to FIG. 1, after a cancerous (or precancerous) breast mass has been detected and removed via surgery 10, the removed tissue is examined for its size. Volumetric displacement of the tissue 12 is used to determine the overall size of the mass. The mass is placed in a known amount of liquid such as water or saline and the change in the volume is determined. This change represents the overall volume of the mass and provides an estimate of the size of the cavity in the breast. Examining the shape 14 is done to determine if the removed mass is uniform in shape or has an unusual shape. Ultrasound or MRI can also be used to determine the shape of the cavity or to verify the shape based on visual examination of the removed mass.

If the shape of the cancerous mass is uniformly spherical or oval shaped, one or two temperature sensors are used. Each sensor is placed 20 by the use of an imaging modality 18 to guide its placement. The sensor will monitor temperature in the tissue at a distance of one centimeter from the cavity. The imaging modality is ultrasound and is well known to those skilled in the art. An alternative imaging modality is stereotactic mammography which is also well known to those skilled in the art. The chosen distance can range from one-half centimeter to two centimeters from the edge of the cavity with one centimeter being preferred.

With unusual, non-uniform shapes, multiple temperature sensors can be used and as many as five sensors on a single probe or up to five sensors on separate probes will be placed 20 at the selected distance and location around the cavity. Each sensor is placed at different positions around the irregularly shaped cavity, all located at a distance of one centimeter from the cavity edge. Alternatively a single probe with two to five temperature sensors is placed with the outer sensors placed at a distance of one centimeter from the cavity's edge and the inner sensors (if more than two) placed at a variable distance from the cavity's edge.

Alternatively, markers may be used in combination with the ultrasound imaging to provide guidance for the probes. The shortest skin-to cavity route is preferably selected for placement of the thermal sensor(s) and the catheter and any intervening vessels are avoided during placement, although this is not critical for the post-surgical technique. Field anesthesia around the cavity is achieved with a long lasting local anesthetic, preferably 0.25% bupivacaine (approximately 50 cc); a single injection is sufficient for the procedure. An additional injection of anesthetic can be made if the patient begins to feel pain during the procedure.

The thermal needle sensor(s) made of surgical grade stainless steel (Omega Engineering, Stamford Conn.) is placed through an incision in the breast and connected to the sensor's monitor. Alternative needle-type thermal sensors include the D-N12 series needle temperature probes made of stainless steel (Exacon Scientific, Copenhagen, Denmark) although any appropriate needle-type thermal sensor can be used for this purpose. Ultrasound is used to confirm proper position of the thermal sensor(s).

A suitable catheter system is the ThermaChoice® II or ThermaChoice® III catheter system and controller (GyneCare WorldWide, Somerville, N.J.). Another catheter system contains sensors for measuring temperature and a heating element internal to the balloon. In another embodiment, the catheter system has a sensor for determining the pressure on the balloon when inflated. The balloon or bladder is strong enough to resist puncture under high temperature and pressure yet may be sufficiently thin to allow even heat flow from the liquid to the breast tissue. All sensor elements are connected to the monitor included with the catheter system.

After the temperature sensor needle(s) and the catheter have been placed, the balloon is filled with a liquid capable of providing even heat transfer from the balloon to the tissue. A suitable liquid is 5% Dextrose saline. Under the chosen imaging modality, the bladder is filled with 5% Dextrose saline until it has molded to the shape of the cavity. The predetermined volume of the cavity is used to determine the fill volume 24. Alternatively, the pressure sensor attached to the catheter system can be used to identify a pressure of 50 to 150 mmHg as the maximum fill 25, with a pressure of 100 mmHg, for example. Imaging using ultrasound, mammography, stereotactic mammography, MRI can be used to verify that the balloon has molded to the shape of the cavity.

After the balloon has been filled, a current is applied 26 to the heating element in the catheter and the fill liquid is heated to a temperature ranging from 80° C. to 95° C. (185° F. to 203.0° F.). After the temperature has reached the target, it is held constant until the temperature of the thermal sensor(s) placed at one centimeter from the cavity has reached 60° C. 28. The thermal sensors are continuously monitored until this temperature has been achieved on all sensors. This is to destroy the tissue surrounding the cavity, including any transformed cells that were missed by the surgery.

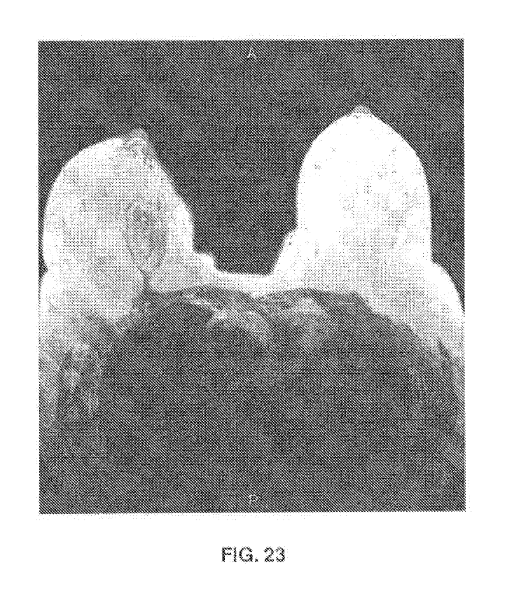

After reaching 60° C., the current is turned off, the fill liquid is emptied from the catheter and the device is removed. The temperature probes are removed and one or more biopsies of the treated area are taken to histologically confirm the impact of the heat on the breast tissue. The patient is thereafter discharged with icepack on the breast and oral analgesic according to the standard operating procedures of the medical facility. A follow up examination 29 is performed to determine the efficacy of the procedure and specifically to verify that the targeted breast tissue has undergone necrosis. Imaging using contrast ultrasound, MRI (FIG. 23), or mammogram can reliably demonstrate the destruction of the tissue surrounding the cavity.

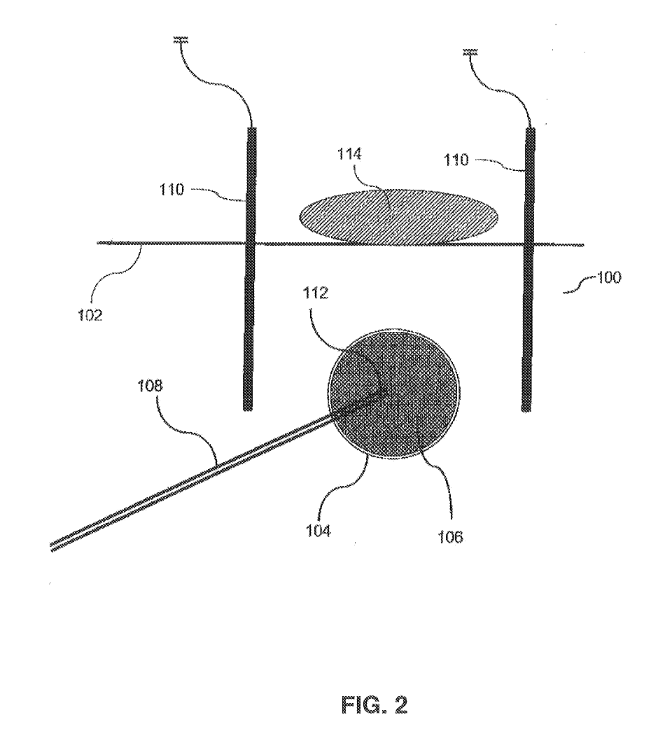

FIG. 2 provides a cross sectional view of the catheter and balloon in the cavity with two temperature sensors placed within the one centimeter radius of the cavity. In this case, the breast 100 with skin surface 102 shown has a cavity 104 located approximately one centimeter in depth from the skin 102. The catheter 108 and thermal balloon 106 have been placed via an incision in the skin 102 so the balloon 106 can be filled with 5% Dextrose saline via a port (not shown) in the catheter 108 to fill the cavity 104. Pressure and temperature sensors (not shown) in the tip 112 of the catheter 108 are used as described to monitor the fill volume pressure and the temperature of the fill fluid. A heating element (not shown) located in the tip 112 of the catheter 108 is used to heat the fill fluid. Additional temperature sensors 110 are placed at one centimeter from the edge of the cavity 104. When the cavity 104 is near the skin 102 (defined as the cavity edge being within two centimeters of the skin), a cold pack 114 can optionally be used at the surface of the breast to regulate heating damage near the skin surface.

### Controller

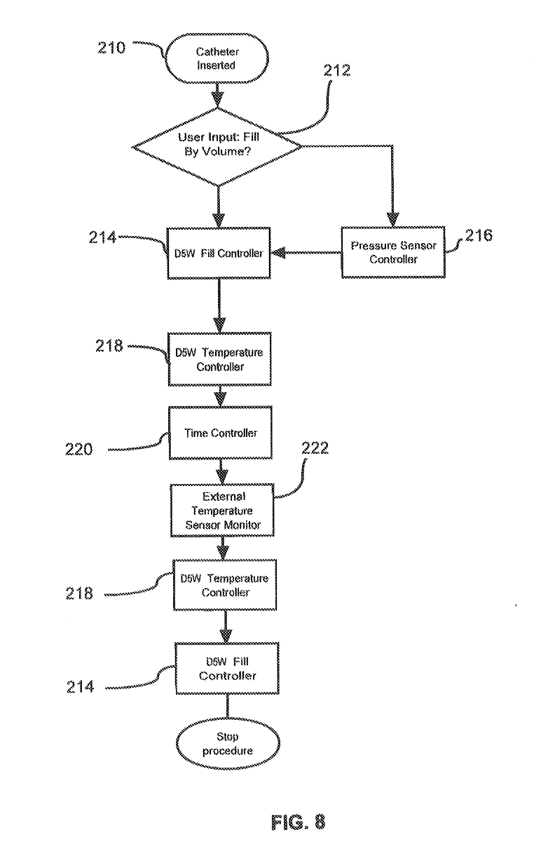

Controller FIG. 8 shows the following steps: after the catheter has been inserted into the post-surgical cavity 210, the user inputs into the controller 212 instructions to fill the catheter by volume (for tissue masses that have uniform shape and have been measured using volumetric displacement as described) or to fill the catheter until a determined pressure has been obtained. If pressure is used to determine the fluid fill, a pressure sensor controller 216 attached to the pressure sensor in the catheter will be set to the desired pressure. The device then begins heating under the guidance of Temperature Controller 218 that is linked to the heating element and to the balloon temperature sensor in the catheter. After the preferred temperature has been reached, a Time Controller 220 activates to monitor the time of the procedure. In the preferred method, one or more External Temperature Sensor Monitor(s) 222 is linked to the temperature sensors and monitors the desired temperature reached at a preferred distance from the balloon. The Time Controller 220 can be optionally set to override the External Temperature Sensor Monitors 222 and shut the procedure down if the procedure extends to a set override time point. After the External Temperature Sensor Monitor(s) placed around the balloon has reached the desired temperature, the device signals Temperature Controller 218 to turn off the heating element and stop heating the fluid. The fill Controller 214 is then signaled to drain the balloon in preparation for its removal.

The targeted 1 cm zone of ablation, surrounding the lumpectomy cavity, may be achieved by heat propagated from a hot balloon placed in the lumpectomy cavity. The balloon, inflated with 5% dextrose solution, is very similar to the Mammosite balloon except for the radium seed being replaced with an electrical element which heats the fluid to 80-95 degrees Celsius and maintains it at that level for a predetermined length of time. The device (ThermaChoice®, by Ethicon) is clinically used for treatment of painful uterine dysfunction. It was selected for this project primarily to test the concept.

### EXAMPLES

### Example 1 Thermal Ablation of the Goat Mammary Gland as a Model for Post-Lumpectomy Treatment of Breast Cancer

### Background

Partial breast irradiation (PBI) post-lumpectomy for carcinoma deploying the Mammosite balloon catheter is practiced as alternative to the whole breast irradiation in selected patients. A one centimeter shell of targeted breast tissue surrounding the lumpectomy cavity may be effectively treated with a balloon inflated with hot fluid instead of irradiation.

Methods:

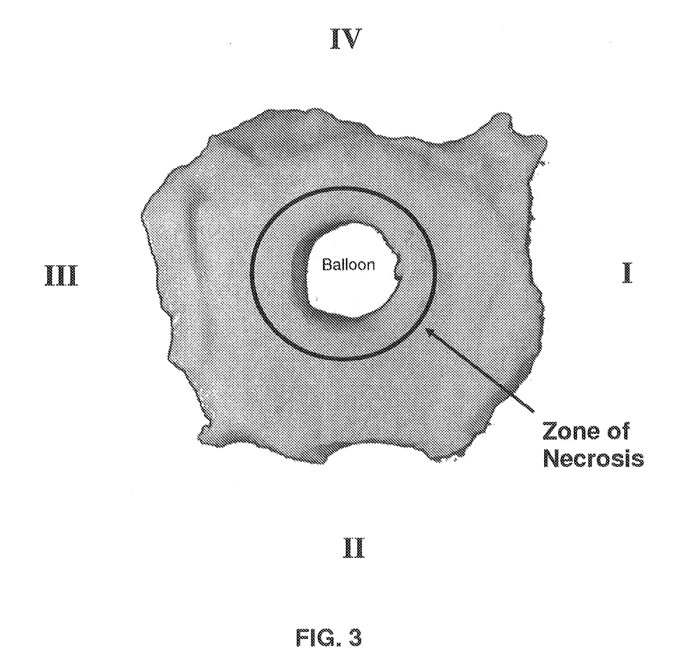

45-50 kilogram Nubian-Cross goats in post-partum phase were selected. Under endo-tracheal anesthesia, the mammary glands measuring 4-7 cm in diameter were visualized by ultrasound and 2-3 cc of the gland were excised for histological documentation and creation of a space for the balloon. A ThermaChoice® deflated balloon (Ethicon) was inserted into the space and the incision was closed ensuring one cm thick tissue coverage. The balloon was inflated with 15-20 cc of 5% dextrose and pre-heated to 87° C., as seen in FIG. 3. At this temperature, mammary glands were treated for periods of 8, 16 and 24 minutes in successive animals that were euthanized 5 or 10-20 days later. The treated glands were excised, serially sectioned perpendicular to the axis of the catheter at 4-5 mm intervals and the necrotic thickness from the point of balloon contact outwards was microscopically measured at 4 points per each section, as seen in FIG. 7. The mean value in mm of the necrosis was recorded against the treatment time in minutes. A total of 14 goats were utilized in this experiment; the first four were used for technical development. The reported data are based on the observations on the last 10 goats.

Eight animals were in the experimental group and two were used as controls.

| GOAT | amount of thermotherapy |
|---|---|
| #1 | 2 minutes |
| #2 | 4 minutes |
| #3 | 6 minutes |
| #4 | 8 minutes |
| #5 | 10 minutes |
| #6 | 12 minutes |
| #7 | 14 minutes |
| #8 | 16 minutes |
| #9 and 10 | control group |

To obtain an 80% chance of detecting a 20% difference in the size of tissue damage and necrosis between the samples from untreated and treated groups, at least 10 samples from each group are needed to achieve a 0.05 significance level in an ANOVA test or student test.

Adult female goats weighing approximately 50 kilograms were used. Under sterile conditions, supine position and general endotacheal anesthesia by glycopyrrolate 0.005-0.01 mpk SQ; xylazine 0.03-0.05 mpk IV followed in 3 minutes by 2-3 mpk ketamine IV. The skin of the mammary area was prepared with Povidone iodine wash and alcohol rinse×3. An elliptical 2×5 cm incision was made adjacent to the first nipple and 30-50 cc of the breast tissue (equivalent of a lumpectomy in patients with operable breast cancer) was removed using sharp dissection. The volume of the excised tissue was measured in a graduated cylinder containing saline and recorded. The deflated balloon was passed into the surgically created space through a small stab incision and was inflated with 30-50 cc of 5% dextrose. With ultrasound guidance the thermal sensor needles were inserted into the tissue 1 cm from the balloon surface. The incision was closed with 3/0 Proline. The balloon surface was at least 1 cm away from the skin, whose temperature will be monitored and if that exceeds 40 Celsius, the skin was cooled with a cold pack.

### Thermal Therapy

The fluid in the balloon was heated to 95° C. using the balloon device. The actual treatment commences after the temperature reaches 95° C. and finished promptly at 2 minute intervals. The balloon was deflated and was removed from the animal. Sterile dressing with an adhesive was applied to the incisions. An identical operation was performed on the second mammary gland of the goat at the next time interval.

The animal was then awakened, extubated and transferred to the recovery room. Over the next 48 hours, each goat was given antibiotic naxcel at 1.1 mg/kg body weight once a day and carprofen at 2-4 mg/kg im.

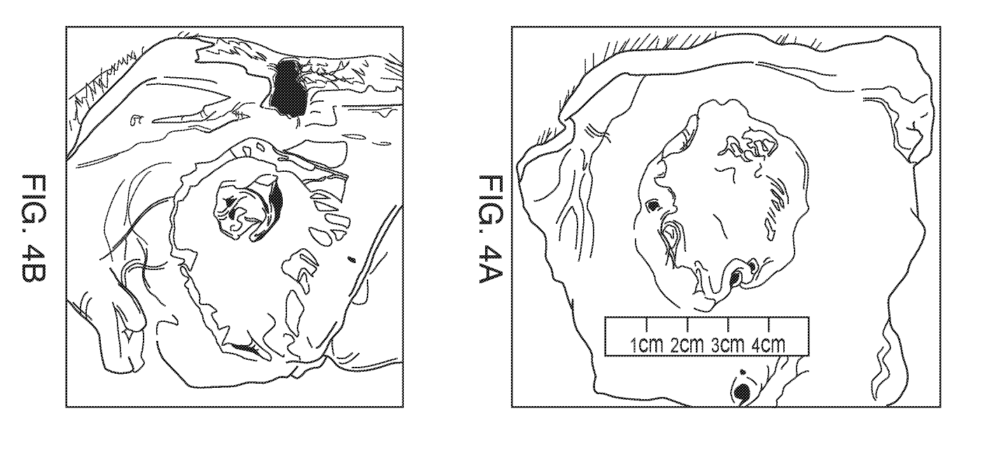

Eight animals were tested, starting at 2 minutes for the first one; the duration of the heat treatment was incrementally increased at 2 minutes. Thus the eighth goat was treated for 16 minutes. At each stage, the treatment was given to 2 animals in both mammary glands for a total of 4 observations. Forty eight hours later, the animals were euthanized and the thermally targeted tissue excised for pathologic examination. The depth of necrosis in millimeters, as defined by a hyperemic zone between viable and devitalized tissue, was measured, as seen in FIG. 5. Sections of the mammary tissue were examined by pathology. A total of ten goats—eight for testing, and two for controls—were needed.

Analysis of the data includes pathologically measuring the thickness in millimeters of tissue necrosis versus the duration of time in the thermally treated mammary tissue presented as a graph and the time it takes to reach the maximal effect of heat on the surrounding mammary tissue of the goat (plateauing of the thermal curve). This was presented as a graph.

The device: ThermaChoice®, manufactured by Ethicon, was programmed to operate at 87 degree Celsius and 150 mm Hg for 8 minute cycles. The inflexibility of the program did not allow the operator to collect data for the first 8 minutes to plot the early portion of the thermal curve to determine the necrosis.

The balloon pressure was fixed to reach 150 mm Hg before the ablation process was commenced. This is appropriate for a muscular uterine application and not for the future breast lumpectomy space where a pressure of 20-40 mm Hg is the average figure noted by the inflated Mammosite balloon.

The data points derived for the three temperature observations of 8, 16 and 24 minutes had wide range of 3 to 20 mm ablation (necrotic depth) from the point of contact with the balloon sections of the thermally treated glands were taken perpendicular to the long axis of the balloon and not radially from the point of balloon contact with the tissue to represent circumferential heat spread. Tighter points will demonstrate more uniform heat propagation unless there is a significant heat sink in the area such as an artery.

Results:

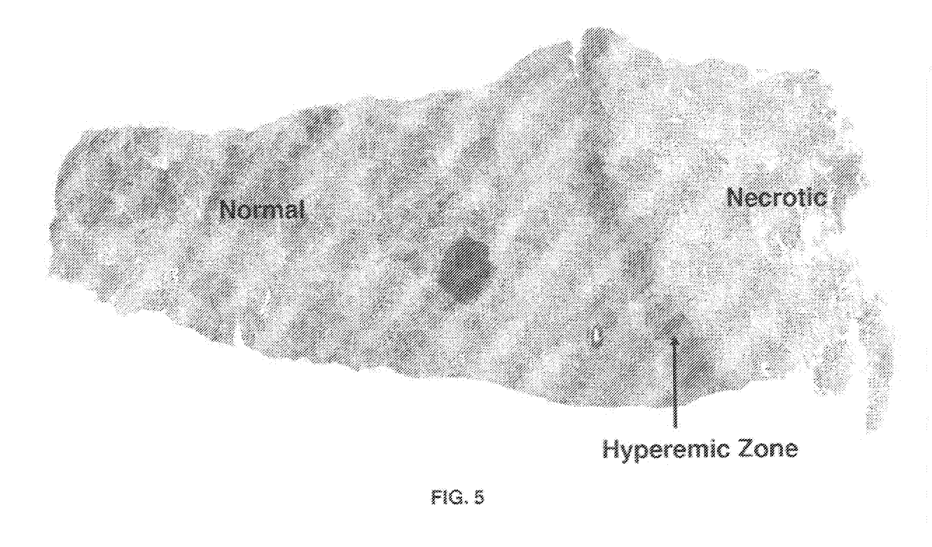

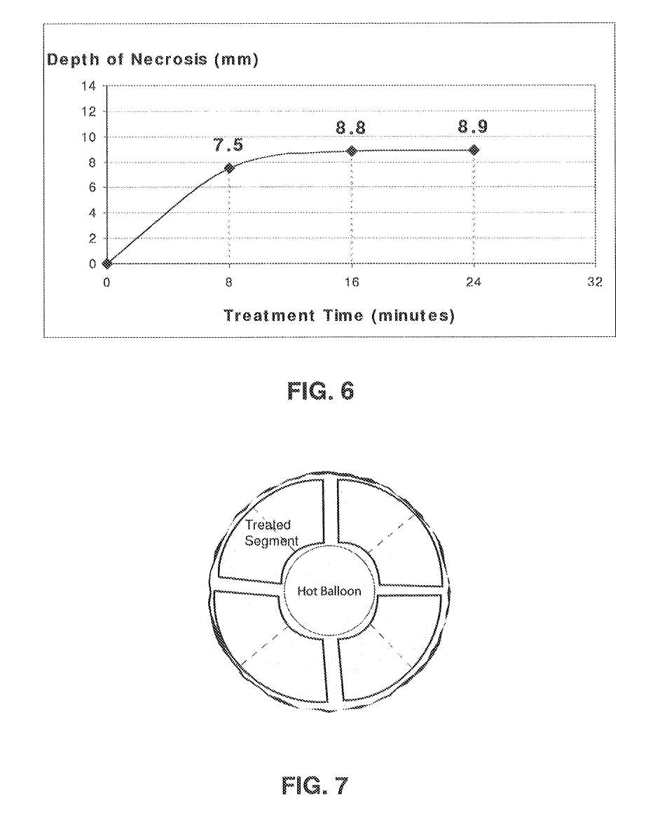

Acute circumferential necrosis of the mammary gland was noted extending from the point of balloon contact with the mammary gland outwards. The data were taken from 20 thermally treated mammary glands in 10 goats. The mean necrosis depth (mm) was tabulated against time in minutes. The depth of necrosis ranged from 3-20 mm with the mean of 7.5, 8.8 and 8.9 mm for 8, 16, 24 minutes of treatment time respectively, as seen in FIG. 6. The programmatic restrictions (electronic controller) of the device such as the time cycles and the high balloon pressure designed for endometrial therapy precluded optimal heat delivery to the mammary gland. Nevertheless, 20 mm zone of necrosis was noted in several instances in different animals.

### CONCLUSION

A model for breast heat therapy is the goat mammary gland. The thermal methods and compositions simulating lumpectomy in human patients, predicts success. Clinical trials will use heat therapy first, and a CT scan to check for tissue damages surrounding the surgical cavity in the breasts, prior to proceeding with standard radiation therapy.

### Results of Heat Therapy, Goat Mammary Glands (see FIG. 6)

| | | Mean (Range) Necrosis |
|---|---|---|
| Treatment Time (mins) | Number of Sections | in mm |
| 8 | 81 | 7.5 (3-20) |
| 16 | 69 | 8.8 (4-15) |
| 24 | 59 | 8.9 (4-17) |

### Example 2: Necrosis in a Cow Udder by Thermal Therapy

Fresh, resected bovine udders were obtained from a local slaughterhouse and tested ex vivo using the thermal therapy system. A deflated balloon was passed into the tissue through a small stab incision and was inflated with 30-50 cc of 5% dextrose in water. Thermal sensor needles were inserted into the tissue 1 cm from the balloon surface. The fluid in the balloon was heated to 95° C. The actual treatment commences after the temperature reaches 95° C. and finished promptly at 2 minute intervals. After the temperature of the sensors reached 60° C., the heating element was turned off and the fluid removed. The deflated balloon was then removed from the tissue. A deep incision was made in the tissue to the point of contact between the balloon and the tissue and the cavity was examined visually and under the microscope.

Analysis of the data included measuring the diameter of the cavity and measuring the thickness of tissue necrosis versus the duration of time in the thermally treated mammary tissue. Pathological examination was performed to examine necrotized versus normal tissue. Acute circumferential necrosis of the tissue was noted extending from the point of balloon contact with the mammary gland outwards.

### Example 3: Experimental Model on a Stereotactic Table

A sheep udder placed in a sealed plastic bag was firmly held within the compression plates of the stereotactic table. Initial scout view was taken and a spot in the center of the udder was selected.

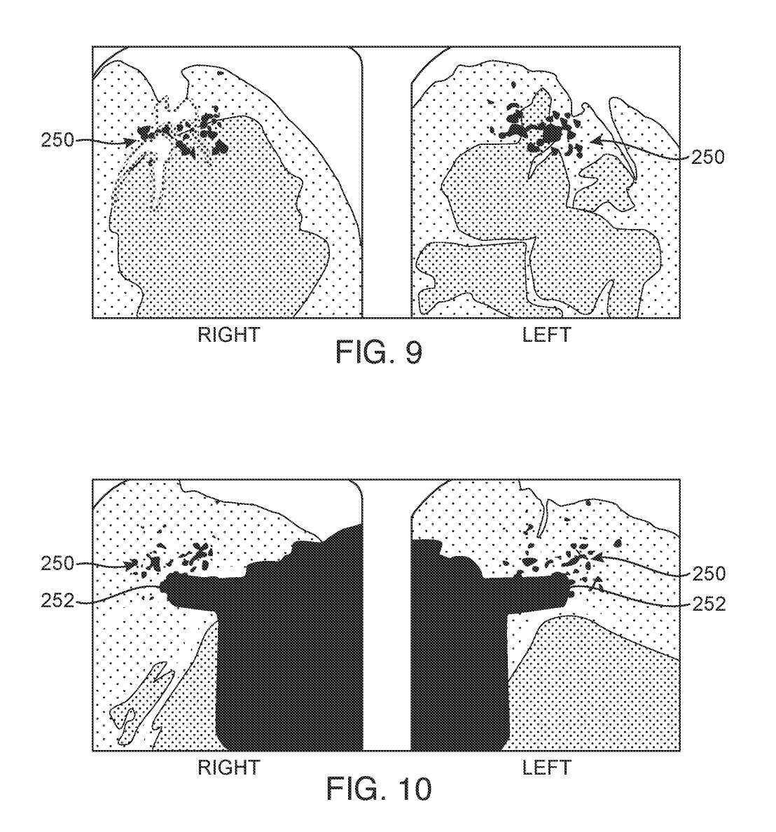

A powder was then created by boiling an egg, removing the shell, and crushing the shell into a powder. The powdered egg shell was then inserted into the sheep udder through an incision and the incision was thereafter closed. The powdered egg shell 250 can be seen in FIG. 9, which depicts images of the sheep udder taken at angles of 15 degrees from right and left sides of the stereotactic table. The images discussed with respect to this example were all taken in a similar manner. The powdered egg shell simulates microcalcifications seen in mammography of human breast as early sign of developing breast cancer. Again, a scout view of the “target” followed by stereo images were taken to determine its dimensions and coordinates within the udder (FIG. 1).

A point immediately below the “target” was chosen, its coordinates are determined and a Mammatome probe was inserted into the udder through a 5 mm incision. Stereo-tactic images were taken, as seen in FIG. 10.

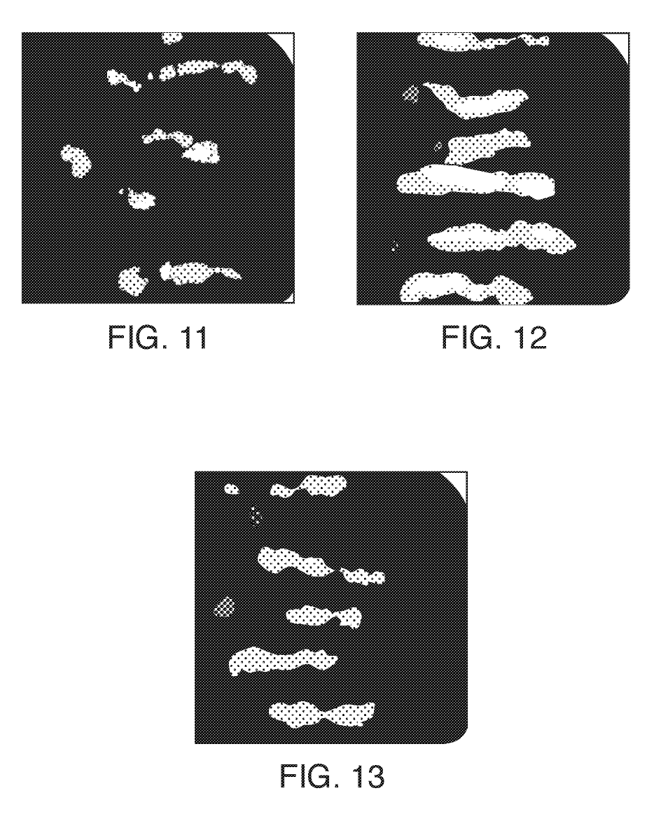

The Mammatome probe 252 was activated ×1 and the “target” was removed and the samples were placed within a container to measure the volume and to be x-rayed. A pair of stereo-images was taken to evaluate the “target”. The samples of the “target” are depicted in FIG. 11 which depicts samples that have a number of cancerous cells.

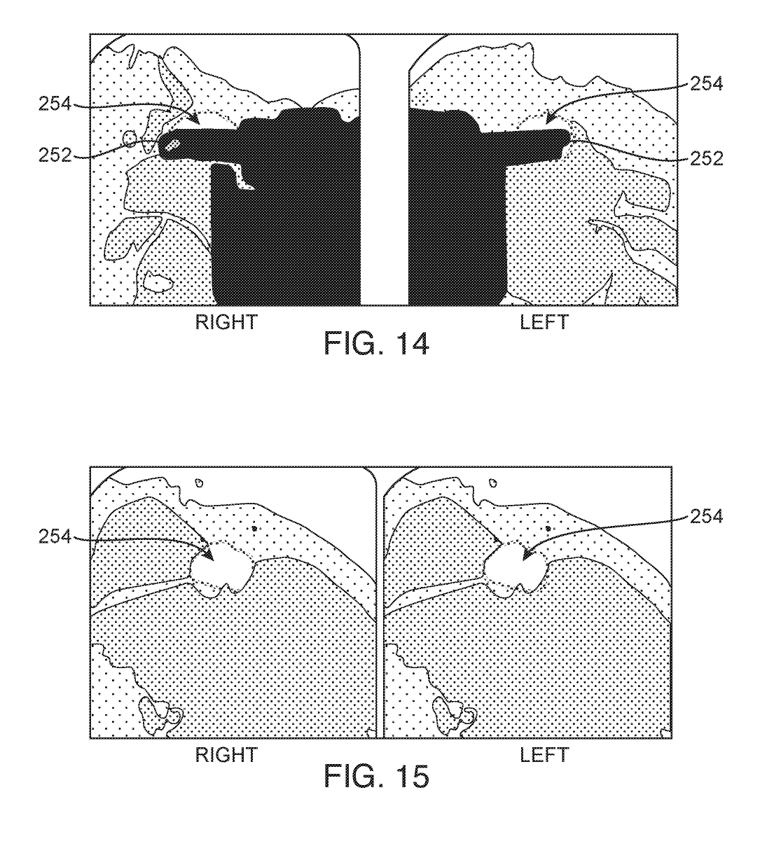

The previous step was repeated 1-2 times. During the first repetition of the previous step, samples were taken, as seen in FIG. 12, in which some cancerous cells were still present. The samples shown in FIG. 12 indicate that, as more tissue in the breast was removed, the remaining tissue became less cancerous. During the second repetition of the previous step, samples were again taken, as seen in FIG. 13. The samples of FIG. 13 indicate that the cancerous cells within the breast were removed. After the removal step was performed three times, another pair of stereo-images was taken, as seen in FIGS. 14 and 15, to depict a cavity 254 left by removal of tissue (FIG. 14 depicts the cavity prior to removal of the probe 252 and FIG. 15 depicts the cavity after removal of the probe 252).

When all the powdered egg shell residue was removed, the created space was measured on the screen and by the volume of the pieces removed by the probe.

Through the incision, the ThermaChoice® probe 256 was inserted into the udder so that its tip comes to lie within the created space; the site of the “target”. The probe's balloon 258 was inflated with dextrose/saline equal to the volume of the tissue removed and until its pressure reaches 50 mm Hg as shown on the device monitor. Stereo-images were taken to confirm satisfactory positioning, as seen in FIG. 16.

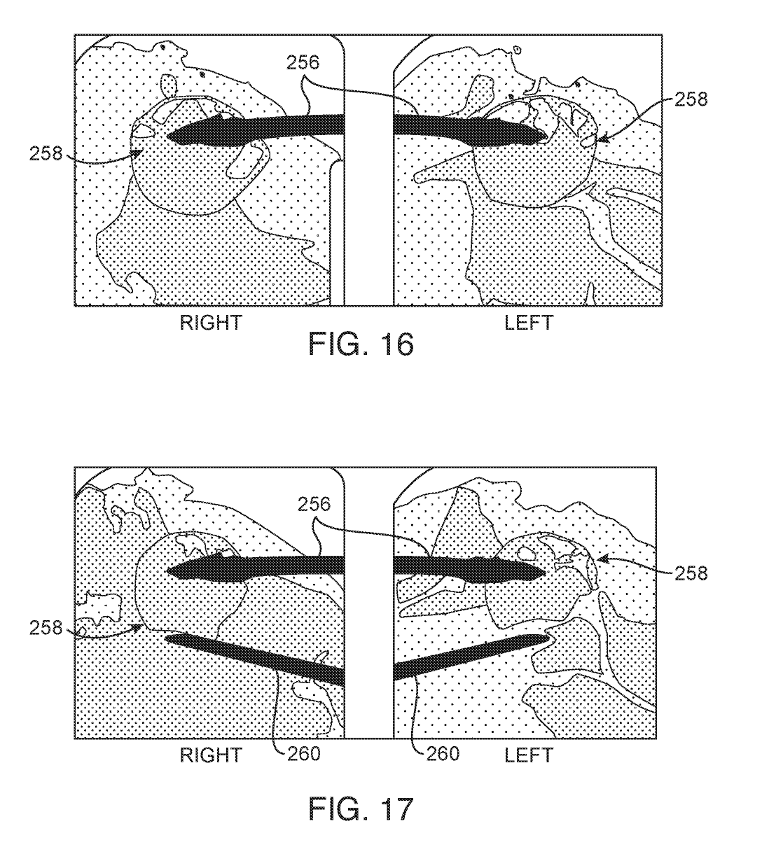

Next, a thermal sensor needle 260 was inserted into the udder 1 cm lateral to the border of the balloon 258 to a depth equal to that of the inserted catheter. Again stereo-images were taken to document precise relationship between the balloon and the thermal needle, as seen in FIG. 17.

The heating element of the Themachoice® probe 256 as activated. After the fluid in the balloon 258 reached 90° C., the therapy time commenced and lasted until the thermal sensor 260 located 1 cm from the balloon records 60° C. The heating was then switched off and stereo-images were taken.

The udder was released from the compression plates without removing the probes. Serial sections of the udder were made parallel with the probes to document the expected tissue color change (yellow to pink) due to heat. Samples of these sections were taken for microscopic examination.

### Example 4: Image-Guided Thermal Therapy of Breast Cancer

Annual screening mammography has resulted in steady size reduction of detected breast cancers. Some of these sub-centimeter tumors appear as nodular densities and others as tissue distortions with or without micro-calcification. During the past two decades, image-guided needle biopsy, employing stereotactic, ultrasound and more recently MRI, has generally replaced open biopsy for diagnosis. Prior to the disclosed thermal therapy, ultrasound technology is used to view the breast of the patient and the cancerous cells or tumor within the breast. A 5-7 mm skin incision is made under local/regional anesthesia and a vacuum-assisted needle/probes is inserted through the skin incision by a medical practitioner to remove the cancer completely as verified by subsequent pathologic examination. The practitioner is able to view and guide the needle/probe using the ultrasound technology. Image-guided removal of sub-centimeter breast cancers allows for the removal of such breast cancers without a major surgery. After removal of the breast cancer, the created space at the site of the tumor can be precisely measured by the volume of the tissue removed from the breast.

The mechanically created channel between the skin and the tumor site stays open for a few days allowing the introduction of a 5 mm catheter after the pathology report indicates complete removal of the cancer. For example the catheter (ThermaChoice®, Ethicon) has a special balloon at its tip which can be inflated with 5% dextrose/saline solution when it reaches the created space within the breast as confirmed by ultrasound or stereotactic images. Use of the balloon for ablation of any cancerous cells surrounding space from which the tumor was removed is described in detail above.

After it is inflated, the balloon has a heating coil located at its base, a thermal sensor to continuously record the fluid temperature and an impeller to mix the heated fluid. Additionally, the balloon has a pressure sensor which activates a heating coil after the fluid pressure reaches at least 50 mm of Hg. Previous experiments with goat mammary glands indicate that the maintenance of this pressure is needed for optimal contact between the balloon and the surrounding breast tissue.

The temperature of the breast tissue is monitored with a thermal needle inserted into the breast parallel with the catheter and 1 cm away from it. This distance is precisely determined by ultrasound or stereotactic images. The operating temperature of the fluid in the balloon is 90° C. After the temperature of the tissue as recorded by the thermal needle located 1 cm from the balloon surface reaches 60° C., the treatment is terminated.

The balloon is deflated by draining the fluid and the catheter is removed. Final stereotactic images are taken before and after the catheter removal. During the therapy the overlying skin temperature is also monitored with cutaneous sensors. The skin is sprayed with a coolant fluid such as ethylene chloride if its temperature exceeds 40° C.

Optional breast examination with Color Doppler ultrasound as known to skilled operators may demonstrate the loss of blood flow to the treated area which may measure about 1 cm.

The image-guided removal and thermal therapy may be used in conjunction with a system including hardware and/or software. The system may include any number of computers, hand-held computers, or any other computing devices. Program code may be implemented within software that is loaded on one or more of the computing devices to operate the image-guided probe. Through the use of ultrasound technology or another suitable technology, the probe and the tissue surrounding the probe may be projected onto a display, for example, a monitor, a hand-held computer, or any other suitable display, in this manner, the doctor guiding the probe is able to view the tumor to be removed and is able to remove such tumor using a non-invasive surgical procedure.

The temperature sensors used during thermal therapy may be operatively connected to the system, for example, to one of the computing devices, by a wired or wireless connection. The system may record data collected from the temperature sensors in a database and may display temperatures, for example, of the fluid within the balloon and/or of the tissue 1 centimeter from the cavity, on a monitor, handheld device, or any other suitable display. The display allows the doctor see the progress of the thermal therapy.

Clinical Experience: Having established its efficacy, the software of the catheter/balloon was modified to adapt to breast treatment after surgical removal of the cancer. The treatment was offered, on a voluntary basis, with consent, to six patients with recurrent breast cancer who had previously been treated with lumpectomy and whole breast radiation and their only option at the time was mastectomy. Each patient studied the therapy details and was satisfied with the explanations of the investigator, before consenting to undergo a second lumpectomy, sentinel node biopsy and to receive thermal therapy of the lumpectomy site while under anesthesia in the operating room.

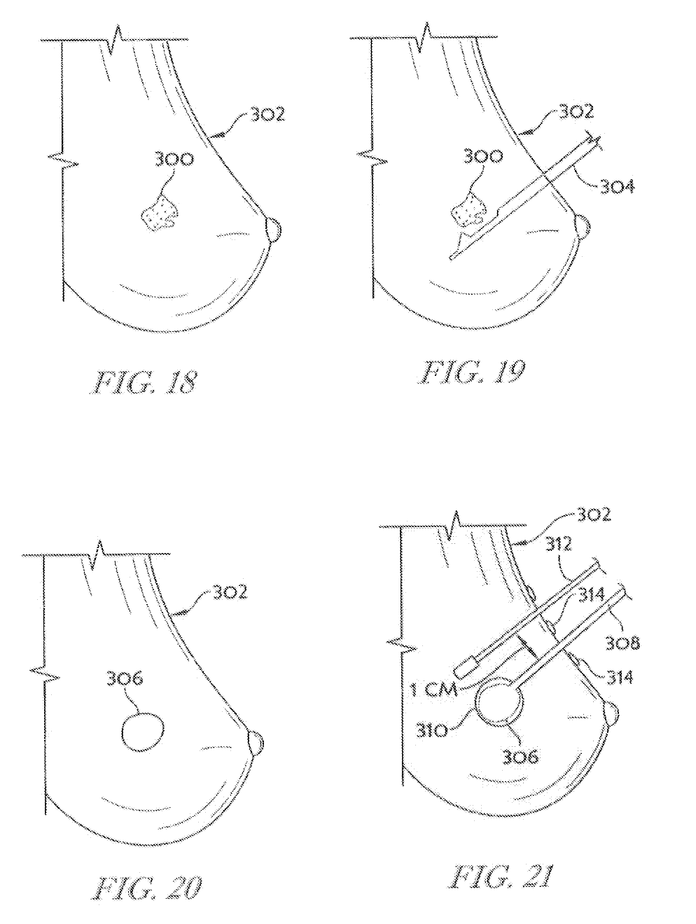

Technical Details: The lumpectomy site prior to removal is depicted in FIG. 18. In particular, cancerous cells or a tumor 300 are shown within a breast 302 of a patient. FIGS. 19 and 20 depict the cancerous tissue 300 being removed by a probe 304 or other surgical tool and the lumpectomy site or cavity 306 after removal, respectively. As seen in FIG. 21, a catheter 308 was then brought into the lumpectomy site 306 through a small peripheral incision of the breast skin and inflated to the volume of the excised cancerous cells or tumor. The catheter 308 (ThermaChoice®, Ethicon) has a special balloon 310 at its tip which can be inflated with saline solution when it reaches the created space within the breast as confirmed by ultrasound or stereotactic images. Once inflated, the balloon 310 has a heating coil located at its base, a thermal sensor to continuously record the fluid temperature and an impeller to mix the heated fluid. Additionally, the balloon 310 has a pressure sensor which automatically activates the heating coil once the fluid pressure within it reaches 40-50 mm of Hg. Previous experiments with goat mammary glands indicate that the maintenance of this pressure is essential for optimal contact between the balloon 310 and the surrounding breast tissue.

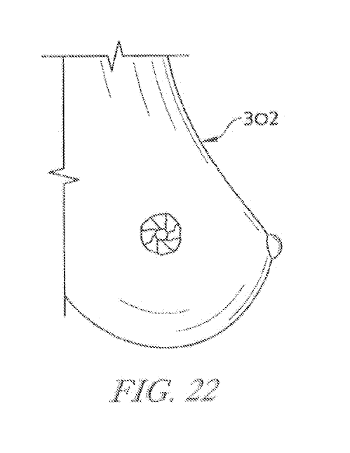

The operating temperature of the fluid in the balloon was 90° C. After the temperature of the tissue as recorded by the thermal needle or sensor 312 located 1 cm from the balloon 310 surface reached 60° C., the treatment was terminated. The balloon 310 was deflated by draining the fluid and the catheter is removed. Final stereotactic images are taken before and after the catheter removal, as seen in FIG. 22. During the therapy, the overlying skin temperature was also monitored with cutaneous sensors 314 (FIG. 21). The skin was sprayed with a coolant fluid such as ethylene chloride if its temperature exceeded 40° C.

Results: The depth of necrosis as measured on biopsies taken immediately from the lumpectomy site ranged 6-14 mm. This was also demonstrated on breast MRI taken within 48 hours of treatment. Transient skin blister was noted in the first two cases necessitating shorter (2 instead of 8 minutes) treatment. No systemic morbidity or recurrence of cancer has been recorded in these patients over 24-54 months of close follow up.

Other objects, features and advantages of the present materials and methods disclosed will become apparent from the following detailed description. It should be understood, however, that the detailed description and the specific examples, while indicating preferred embodiments are given by way of illustration only, since various changes and modifications within the spirit and scope of the disclosure will become apparent to those skilled in the art from this detailed description.

### PUBLICATIONS CITED

Dickler et al., Intl. Jrnl. Radiation Oncology & Biological Physics, Vol 59, No 2 pps 469-474
Dowlatshahi et al 2002), Am J. Surgery, vol 184 pps 359-363

1. A method of removing undesirable cells and killing cells surrounding a cavity caused by the removal of the undesirable cells, the method comprising the steps of: (a) inserting a probe through an incision made in the skin of a patient; (b) removing undesirable cells using the probe and using image-guided technology; (c) inserting a balloon into the cavity formed by the removal of the undesirable cells; (d) conforming the balloon to a shape of the cavity and providing sufficient fluid to fill the cavity based on the volume; and (e) reaching a temperature in the fluid to achieve a temperature in the tissue surrounding the cavity that kills cells surrounding the cavity.

2. The method of claim 1, wherein the temperature that is reached in the fluid is in the range of 80.degree.-95.degree. C.

3. The method of claim 1, wherein the heated tissue is at a temperature of about 60.degree. C. at 1 cm from a surface of the balloon.

4. The method of claim 1, wherein the undesirable cells are in a breast.

5. The method of claim 1, further including the step of filling the balloon with fluid using a catheter.

6. The method of claim 1, wherein the fluid comprises dextrose/saline.

7. The method of claim 1, wherein the incision used for inserting the probe is used for inserting the balloon and providing fluid to the balloon.

8. A method of removing undesirable cells and killing cells surrounding a cavity caused by the removal of the undesirable cells, the method comprising the steps of: (a) inserting a probe through an incision made in the skin of a patient; (b) removing undesirable cells using the probe and using image-guided technology; (c) inserting a balloon into the cavity formed by the removal of the undesirable cells; (d) conforming the balloon to a shape of the cavity and providing sufficient fluid to fill the cavity based on the volume; (e) reaching a temperature in the fluid to achieve a temperature in the tissue surrounding the cavity that kills cells surrounding the cavity; and (f) placing at least one temperature sensor under the skin of the patient and spaced from the cavity.

9. The method of claim 7, wherein the temperature that is reached in the fluid is in the range of 80.degree.-95.degree. C.

10. The method of claim 7, wherein the heated fluid is at a temperature of about 90.degree. C. in the cavity.

11. The method of claim 7, wherein the undesirable cells are in a breast.

12. The method of claim 7, further including the step of filling the balloon with fluid using a catheter.

13. The method of claim 7, wherein the fluid comprises dextrose.

14. The method of claim 7, wherein the placing step includes placing a plurality of temperature sensors under the skin of the patient and spaced from the cavity.

15. The method of claim 7, wherein the at least one temperature sensor is disposed within the tissue of the patient about 1 cm from the cavity.

16. The method of claim 14, further including placing a temperature sensor within the balloon to measure a temperature of the fluid within the balloon.
